(19) United States
(12) Patent Application Publication
(10) Pub. No.: US 2022/0370851 A1
(43) Pub. Date: Nov. 24, 2022

(54) Method and System for Determining Instantaneous Effort Value

(71) Applicant: CITYROW Holdings, Inc., New York, NY (US)

(72) Inventors: Anthony Guidarelli, Brooklyn, NY (US); Jay Chmilewski, Brooklyn, NY (US)

(21) Appl. No.: 17/326,044

(22) Filed: May 20, 2021

(51) Int. Cl. A63B 24/00; A63B 71/06

(52) U.S. Cl. CPC A63B 24/0062; A63B 24/0075; A63B 24/0087; A63B 71/0622; A63B 2024/0065; A63B 2024/0068; A63B 2024/0081; A63B 2024/0093; A63B 2071/063

(57) ABSTRACT

Methods and systems for calibrating a level of intensity of a fitness exercise given a target effort value facilitate optimal exertion for an individual performing the fitness exercise. An instantaneous effort value is determined based on measured real-time and historical data and communicated to the individual. An aspect of the fitness exercise is adjusted based on a difference between the instantaneous effort value and a target effort value.

### BACKGROUND

As fitness instruction and tracking become more accessible, new strategies and techniques for remotely guiding individuals through fitness exercises are required to create the best experience and convey instructional information more effectively.

### SUMMARY

Embodiments provide improved fitness instruction and performance measurement through determination and use of an instantaneous effort value that is easy to understand and that facilitates optimal exertion during exercise.

In one embodiment, a method of calibrating, for an individual performing a fitness exercise, a level of intensity of the fitness exercise given a target effort value includes configuring a processor to determine an instantaneous effort value corresponding to an instantaneous level of physical and mental exertion for the individual. The determining of an instantaneous effort value includes obtaining measured data from an exertion monitor associated with the individual, the measured data including at least measurements of power. The determining of an instantaneous effort value further includes determining performance metrics from the measured data, the performance metrics including at least a peak power value and an average power value. The performance metrics can also include heart rate, respiratory rate or body positioning data. The determining of an instantaneous effort value further includes obtaining historical performance metrics for the individual, the historical performance metrics including at least historical peak power values and historical average power values. The power values may be reflective of power output by an individual, or power input to an exercise machine, or a combination thereof. The power values may be measured, for example, in Watts or calories per hour, or may be a modality-specific power measurement. The historical performance metrics may be obtained from the memory device, or alternatively raw historical performance data may be obtained from the memory device and historical performance metrics may be calculated therefrom.

The embodiment continues such that the determining of an instantaneous effort value further includes creating a best-fit equation. The equation may be based, at least in part, on historical performance data previously obtained from exertion monitors associated with at least one prior individual, which prior individual may be the same or different from the individual presently performing the fitness exercise. The equation may be based, at least in part, on expertise in a specific fitness exercise modality. For example, constants in the equation may be derived from analysis done on the historical performance data. Such analysis may include a comparison between historical performance data of prior individuals, including historical observed estimated effort values, and corresponding desired effort values. The desired effort values may be defined at various values of performance metrics for a given fitness exercise modality, thereby providing expertise in the modality, which expertise may thus be incorporated into the best-fit equation. In other examples, constants in the equation may be personalized or otherwise modified for the individual, modified for a specific fitness exercise modality or workout program, or modified based on other factors specific to a given scenario. Historical performance data may be represented in tabular format. An example of such historical performance data is a power-based split time measurement in rowing. An instantaneous split time value may be an independent variable used in the best-fit equation, and may be used in combination with other performance metrics values.

In the embodiment, the best-fit equation is modeled on a shape of a graph of the desired effort values defined at various values of performance metrics for the given fitness exercise modality. The equation in the embodiment is created by developing reference data based on the historical performance data, and based on expertise in the given modality, which expertise includes the desired effort values. In embodiments incorporating split times for a rowing modality, the shape of the graph is an S-curve, and a logistic regression is used to derive the equation. The best-fit equation is thus optimized in the embodiment based on distributions of historical values and a modality-specific understanding of individual effort in relation to performance metrics. Other independent variables based on other performance metrics can be added to the equation to increase precision. Similar analysis can be completed on historic data of the other performance metrics to determine constants that can be included in the equation. The embodiment continues such that the determining of an instantaneous effort value further includes calculating the instantaneous effort value for the individual using the best-fit equation.

The embodiment continues such that the processor is further configured to communicate, via a display device, the instantaneous effort value to the individual. The processor is further configured to determine a difference between the instantaneous effort value and the target effort value. The processor is further configured to adjust an aspect of the fitness exercise based on the determined difference.

In some embodiments, the exertion monitor may include sensors placed proximal to the individual or to an exercise machine employed by the individual. In some embodiments, the fitness exercise may be one of running, cycling, and rowing.

In some embodiments, the performance metrics may include measurements of cadence. Measurements of cadence may include, for example, steps per minute, revolutions per minute, or strokes per minute. In some embodiments, the performance metrics may include distance-based measurements. Distance-based measurements may include, for example, speed in miles per hour or kilometers per hour, or pace in minutes per mile, minutes per kilometer, minutes per 500 meters, or minutes per 2000 meters. In some embodiments, the performance metrics may include at least one of heart rate measurements, respiratory rate measurements, and body positioning measurements.

In some embodiments, the display device may be disposed proximal to an exercise machine. In some embodiments, communicating the instantaneous effort value to the individual may include indicating, on the display device, a visual representation of the instantaneous effort value, or a time series thereof, during performance of the fitness exercise by the individual. In some embodiments, adjusting an aspect of the fitness exercise may include automatically modifying a level of resistance on an exercise machine. Alternatively, or in addition, in some embodiments, adjusting an aspect of the fitness exercise may include alerting a trainer to advise the individual to modify his or her level of effort. In some embodiments, an exercise machine may be, for example, a rowing machine, a stationary or spinning bicycle, a conventional bicycle, or a treadmill.

In some embodiments, historical performance metrics, including average power values, may be modified by a decay factor such that more recent historical performance metrics are weighted more strongly and less recent historical performance metrics are weighted less strongly in a calculation of the instantaneous effort value. Such weighting may be achieved by using a weighted moving average calculation with a chosen alpha value and a time (t) value measured in number of days. In some embodiments, the method may further include transmitting an audio and video representation of the individual's performance of the fitness exercise to a device of a remotely located trainer, and further comprising transmitting an audio and video representation of a coaching session of the trainer to the display device to be viewed by the individual.

In another embodiment, a system for calibrating, for an individual performing a fitness exercise, a level of intensity of the fitness exercise given a target effort value, may include a processor and a memory device with instructions loaded thereon, the instructions, when loaded, configuring the processor to determine an instantaneous effort value corresponding to an instantaneous level of physical and mental exertion for the individual. The determining of an instantaneous effort value includes obtaining measured data from an exertion monitor associated with the individual, the measured data including at least measurements of power. The determining of an instantaneous effort value further includes determining performance metrics from the measured data, the performance metrics including at least a peak power value and an average power value. The performance metrics can also include heart rate, respiratory rate or body positioning data. The determining of an instantaneous effort value further includes obtaining historical performance metrics for the individual, the historical performance metrics including at least historical peak power values and historical average power values. The power values may be reflective of power output by an individual, or power input to an exercise machine. The power values, for example, may be measured in Watts or in calories per hour, or may be a modality-specific power measurement. The historical performance metrics may be obtained from the memory device, or alternatively raw historical performance data may be obtained from the memory device and historical performance metrics may be calculated therefrom.

The embodiment continues such that the determining of an instantaneous effort value further includes calculating, using a polynomial equation derived from a best-fit analysis applied to a set of historical observed estimated effort values, the instantaneous effort value for the individual based on the determined and historical performance metrics.

The embodiment continues such that the processor is further configured to communicate, via a display device, the instantaneous effort value to the individual. The processor is further configured to determine a difference between the instantaneous effort value and the target effort value. The processor is further configured to adjust an aspect of the fitness exercise based on the determined difference. This embodiment may further optionally include any features described herein in connection with any of the other embodiments described herein.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a flow diagram depicting an example embodiment of a method of calibrating a level of intensity of a fitness exercise being performed by an individual, given a target value.

FIG. 2 is a flow diagram depicting an example embodiment of a method of determining an instantaneous effort value.

FIG. 3 is a schematic block diagram illustrating an example embodiment of a system for calibrating a level of intensity of a fitness exercise being performed by an individual, given a target value.

FIG. 4 illustrates an example computer network, over which, embodiments of the claimed systems and methods may operate.

FIG. 5 is a system block diagram illustrating an example computer network, over which, embodiments of the claimed systems and methods may operate.

### DETAILED DESCRIPTION

A description of example embodiments follows.

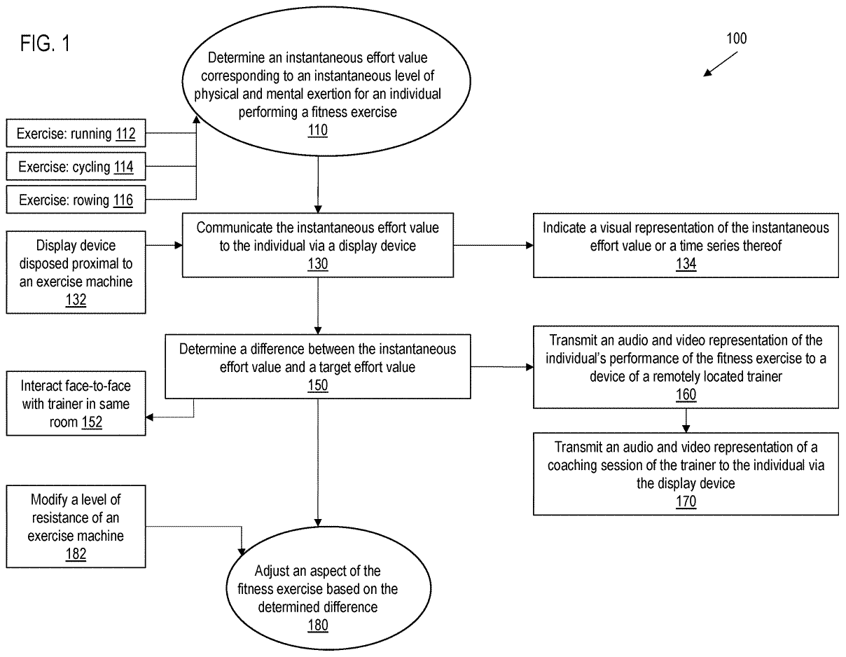

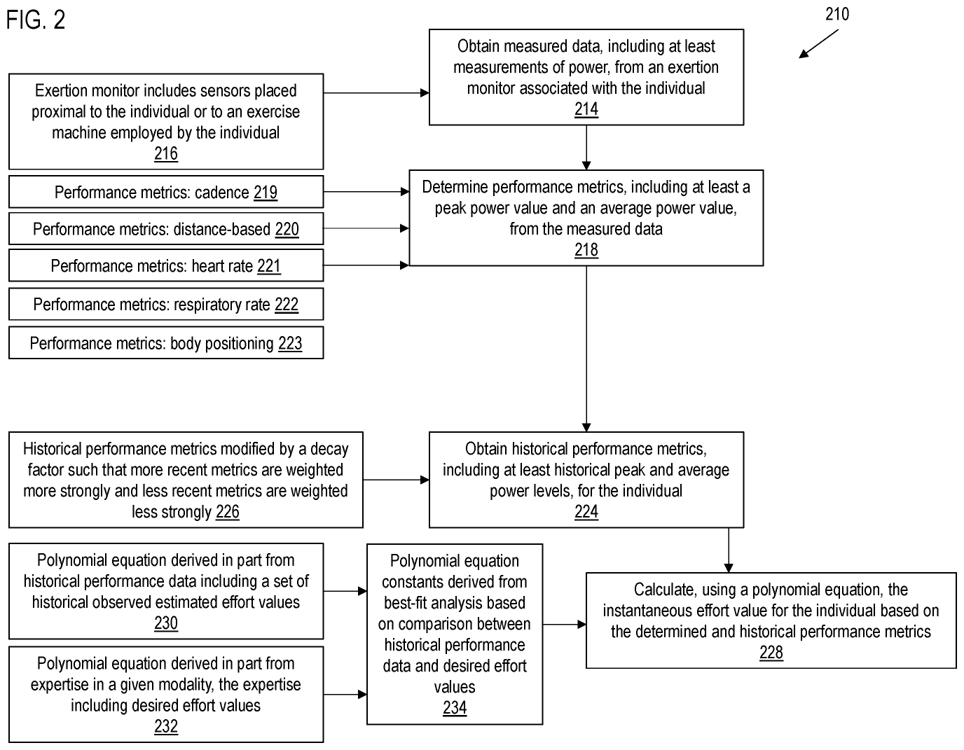

FIGS. 1 and 2 show an example method of calibrating, for an individual performing a fitness exercise, a level of intensity of the fitness exercise given a target effort value. It should be noted that the examples presented herein are not limiting, and that other example methods of calibrating a level of intensity of a fitness exercise may be realized through different combinations of the various elements shown in FIGS. 1 and 2 and described herein.

FIG. 1 illustrates an embodiment of a method 100 of calibrating, for an individual performing a fitness exercise, a level of intensity of the fitness exercise given a target effort value. According to the embodiment, a processor is configured to determine an instantaneous effort value 110 corresponding to an instantaneous level of physical and mental exertion for an individual performing a fitness exercise. Mental exertion may be understood as a level of concentration, or active consideration of various mechanical details of execution of an exercise. As physical exertion is increased, an individual may have a natural tendency to perform the exercise with a deteriorating level of form or technique due to fatigue or another physical limitation. As such, a corresponding increase in mental exertion may be required to preserve a consistent level of form or technique. The exercise may be one of running 112, cycling 114, and rowing 116. The processor is further configured to communicate 130 the instantaneous effort value to the individual via a display device. The display device may be disposed 132 proximal to an exercise machine. The communicating 130 may include indicating 134, on the display device, a visual representation of the instantaneous effort value, or a time series thereof, during performance of the fitness exercise by the individual.

Continuing with reference to FIG. 1, in the embodiment, the processor is further configured to determine a difference 150 between the instantaneous effort value and the target effort value. The determined difference 150 may be discussed and acted upon by the individual and a trainer interacting with each other face-to-face 152 within the same room or location. The processor may be further configured to transmit an audio and video representation of the individual's performance of the fitness exercise 160 to a device of a remotely located trainer. The processor may be further configured to transmit an audio and video representation of a coaching session of the trainer 170 to the display device to be viewed by the individual. The processor is further configured to adjust 180 an aspect of the fitness exercise based on the determined difference 150 between the instantaneous effort value and the target effort value. The adjusting 180 of an aspect of the fitness exercise may include modifying 182 a level of resistance of an exercise machine. Alternatively, or in addition, in some embodiments, the adjusting 180 of an aspect of the fitness exercise may include alerting a trainer to advise the individual to modify his or her level of effort. In some embodiments, an exercise machine may be, for example, a rowing machine, a stationary or spinning bicycle, a conventional bicycle, or a treadmill.

FIG. 2 illustrates an embodiment of a method 210 for determining an instantaneous effort value 110 corresponding to an instantaneous level of physical and mental exertion for an individual performing a fitness exercise within a context as established above with reference to FIG. 1. In the embodiment, a processor is configured to obtain measured data 214 from an exertion monitor associated with an individual. The measured data 214 includes at least measurements of power. The exertion monitor may include sensors 216. The sensors 216 may be disposed proximal to the individual or to an exercise machine employed by the individual, or a combination thereof. With sensors 216 disposed proximal to an exercise machine employed by the individual, efficiency, for example due to a level of form or technique with which the individual is performing the exercise, may be included as a component of the measured data 214. Quality of form or technique of the individual performing the exercise may thus be incorporated into the measured data 214. The processor is further configured to determine performance metrics 218 from the measured data. The performance metrics 218 include at least a peak power value and an average power value. The power values may be reflective of power output by an individual, or power input to an exercise machine. The power values may be measured in Watts. The performance metrics 218 may include measurements of cadence 219. Measurements of cadence 219 may include, for example, steps per minute, revolutions per minute, or strokes per minute. The performance metrics 218 may include distance-based measurements 220. Distance-based measurements 220 may include, for example, speed in miles per hour or kilometers per hour, or pace in minutes per mile, minutes per kilometer, minutes per 500 meters, or minutes per 2000 meters. The performance metrics may include measurements of heart rate 221. The performance metrics may include measurements of respiratory rate 222. The performance metrics may include measurements of body positioning 223.

The embodiment depicted in FIG. 2 continues as a processor is configured to obtain historical performance metrics 224 for the individual. The historical performance metrics 224 include at least a historical peak power level and a historical average power level. The historical performance metrics 224 may be obtained from the memory device, or alternatively raw historical performance data may be obtained from the memory device and historical performance metrics 224 may be calculated therefrom. The historical performance metrics 224 may be modified by a decay factor 226 such that more recent historical performance metrics are weighted more strongly and less recent historical performance metrics are weighted less strongly in a calculation of the instantaneous effort value. The processor is further configured to calculate the instantaneous effort value 228 for the individual based on the determined 218 and historical 224 performance metrics. The instantaneous effort value is calculated 228 using a polynomial equation. The polynomial equation used in calculating the instantaneous effort value 228 may be derived, in whole or in part, from historical performance data 230 previously obtained from exertion monitors associated with at least one prior individual, which prior individual may be the same or different from the individual presently performing the fitness exercise. The equation may be derived, in whole or in part, from expertise 232 in a specific fitness exercise modality. For example, constants in the equation may be derived from analysis done on the historical performance data. Such analysis may include a comparison 234 between historical performance data of prior individuals, including a set of historical observed estimated effort values 230, and corresponding desired effort values 232. The desired effort values 232 may be defined at various values of performance metrics for a given fitness exercise modality, thereby providing expertise in the modality, which expertise may thus be incorporated into the best-fit equation. In other examples, constants in the equation may be personalized or otherwise modified for the individual, modified for a specific exercise modality or workout program, or modified based on other factors specific to a given scenario. The instantaneous effort value 228 and the historical observed estimated effort values 230 may be expressed as, for example, a percentage. Historical performance data such as the historical observed estimated effort values 230 may be represented in a tabular format. An example of such historical performance data is a power-based split-time measurement in rowing. An instantaneous split time value may be an independent variable used in the best-fit equation, and may be used in combination with other performance metrics values.

In some embodiments, the best-fit equation is modeled on a shape of a graph of the desired effort values defined at various values of performance metrics for the given fitness exercise modality. The equation in the embodiment is created by developing reference data based on the historical performance data, and based on expertise in the given modality, which expertise includes the desired effort values 232. In embodiments incorporating split times for a rowing modality, the shape of the graph is an S-curve, and a logistic regression is used to derive the equation. The best-fit equation is thus optimized in the embodiment based on distributions of historical values and a modality-specific understanding of individual effort in relation to performance metrics. Other independent variables based on other performance metrics can be added to the equation to increase precision. Similar analysis can be completed on historic data of the other performance metrics to determine constants that can be included in the equation.

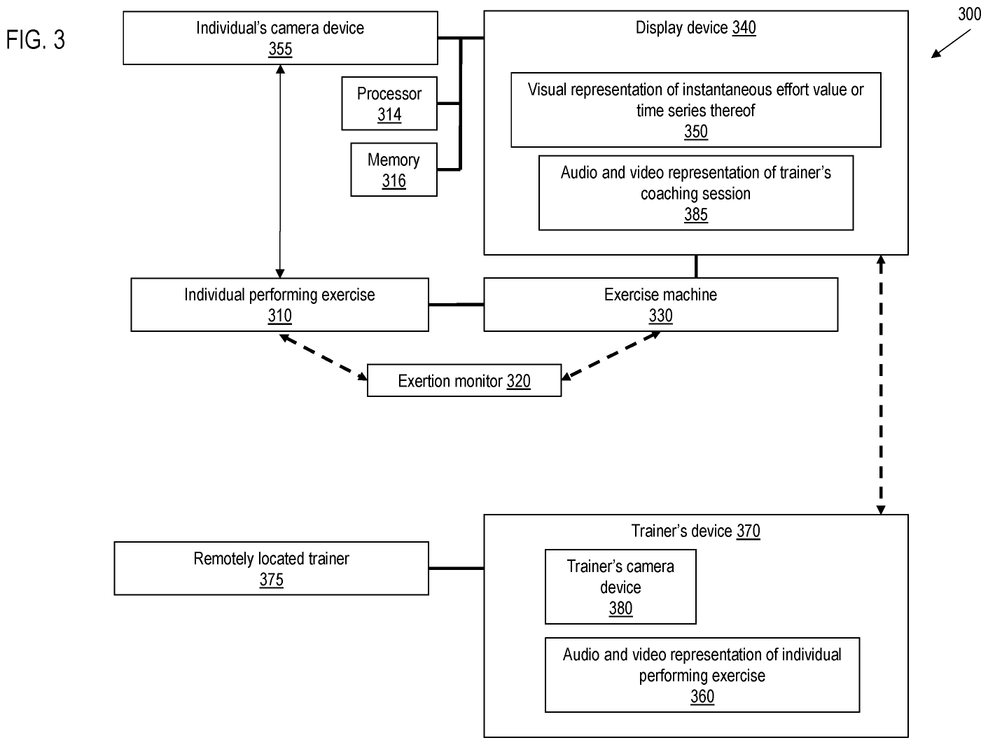

FIG. 3 depicts an example system 300 for calibrating, for an individual performing a fitness exercise 310, a level of intensity of the fitness exercise given a target effort value. The system 300 includes a processor 314 and memory device 316, the memory device 316 loaded with instructions that, when loaded, configure the processor 314 to determine an instantaneous effort value corresponding to an instantaneous level of physical and mental exertion for the individual.

According to the embodiment shown in FIG. 3, the determining of an instantaneous effort value includes obtaining measured data from an exertion monitor 320 associated with the individual 310. The exertion monitor may be disposed proximal to the individual 310, or to an exercise machine 330 employed by the individual 310. The determining of an instantaneous effort value further includes, at the processor 314, determining performance metrics from the measured data, the performance metrics including at least a peak power value and an average power value. In some embodiments, the performance metrics may include at least one of heart rate data, respiratory rate data, and body positioning data. The determining of an instantaneous effort value further includes, at the processor 314, obtaining historical performance metrics for the individual, the historical performance metrics including at least historical peak power values and historical average power values. The power values may be reflective of power output by an individual, or power input to an exercise machine. The power values may, for example, be measured in Watts, or in calories per hour, or may be a modality-specific measurement. The historical performance metrics may be obtained from the memory device 316, or alternatively raw historical performance data may be obtained from the memory device 316 and historical performance metrics may be calculated therefrom. The determining of an instantaneous effort value further includes, at the processor 314, calculating, using a polynomial equation derived from a best-fit analysis applied to a set of historical observed estimated effort values, the instantaneous effort value for the individual based on the determined and historical performance metrics.

The application of a best-fit analysis to a set of historical observed estimated effort values, as described above with reference to FIG. 3, may further include a comparison of the historical observed estimated effort values, or other forms of historical performance data of prior individuals, with corresponding desired effort values. The desired effort values may be defined at various values of performance metrics for a given fitness exercise modality, thereby providing expertise in the modality, which expertise may thus be incorporated into the best-fit equation. The best-fit equation may be modeled on a shape of a graph of the desired effort values defined at various values of performance metrics for the given fitness exercise modality. The equation may be created by developing reference data based on the historical performance data, and based on expertise in the given modality, which expertise includes the desired effort values. The best-fit equation may thus be optimized based on distributions of historical values and a modality-specific understanding of individual effort in relation to performance metrics.

Further to the embodiment shown in FIG. 3, the processor 314 is further configured to communicate, via a display device 340, the instantaneous effort value to the individual. The communicating of the instantaneous effort value via the display device 340 may include presenting, on the display device 340, a visual representation 350 of the instantaneous effort value, or a time series thereof.

Continuing to describe the embodiment depicted in FIG. 3, the processor may be configured to accept, from a camera device 355 of the individual, an audio and video representation of the individual performing the fitness exercise 360. The camera device 355 may be used to collect body positioning data to be used as a performance metric. The processor 314 may be optionally configured to transmit the audio and video representation of the individual performing the fitness exercise 360 to a device 370 of a trainer. The trainer may be a remotely located trainer 375. The processor 314 may be optionally configured to accept, from a camera device 380 of the trainer, an audio and video representation of a coaching session 385 of the trainer. The processor 314 may be optionally configured to transmit the audio and video representation of the coaching session 385 to the display device 340 of the individual.

In FIG. 3, if a trainer happens to be located in the same room as, or otherwise in the vicinity of the individual 310 such that face-to-face communication is practical, a trainer's device 370 may not be present. As such, audio and video representations of the individual performing the exercise 360 and of a coaching session 385 of the trainer may or may not be transmitted.

Further to the embodiment depicted in FIG. 3, the processor 314 is further configured to determine a difference between the instantaneous effort value and the target effort value. The processor 314 is further configured to adjust an aspect of the fitness exercise based on the determined difference. The camera device 355 of the individual may be integrated with, or separate from, the display device 340. The processor 314 and memory 316 may be integrated with, or implemented in a device separate from, the display device 340. The camera device 380 of the trainer may be integrated with, or separate from, the device 370 of the trainer. In some embodiments, an exercise machine may be, for example, a rowing machine, a stationary or spinning bicycle, a conventional bicycle, or a treadmill.

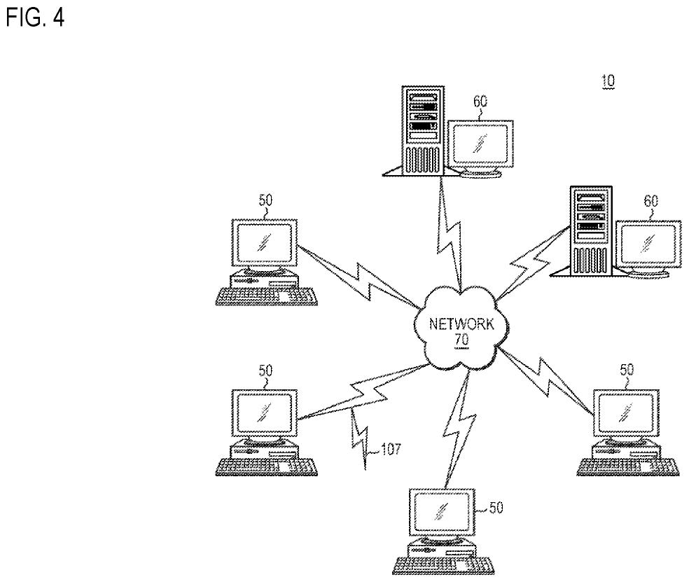

FIG. 4 illustrates a computer network (or system) 10 or similar digital processing environment, according to some embodiments of the present disclosure. Client computer(s)/devices 50 and server computer(s) 60 provide processing, storage, and input/output devices executing application programs and the like. The client computer(s)/devices 50 can also be linked through communications network 70 to other computing devices, including other client devices/processes 50 and server computer(s) 60. The communications network 70 can be part of a remote access network, a global network (e.g., the Internet), a worldwide collection of computers, local area or wide area networks, and gateways that currently use respective protocols (TCP/IP, Bluetooth®, Near-Field Communication (NFC), etc.) to communicate with one another. Other electronic device/computer network architectures are suitable.

Client computers/devices 50 may be configured with a computing module (located at one or more of elements 50, 60, and/or 70). In some embodiments, a user may access the computing module executing on the server computers 60 from a user device, such a mobile device, a personal computer, or any computing device known to one skilled in the art without limitation. According to some embodiments, the client devices 50 and server computers 60 may be distributed across a computing module.

Server computers 60 may be configured as the computing modules which communicate with client devices 50 for providing access to (and/or accessing) databases that include fitness data of an individual, such as performance metrics and an instantaneous effort value. The server computers 60 may not be separate server computers but part of cloud network 70. In some embodiments, the server computer (e.g., computing module) may enable calibrating of a level of intensity of a fitness exercise, for an individual performing the fitness exercise, by allowing access to data located on the client 50, server 60, or network 70 (e.g., global computer network). The client (configuration module) 50 may communicate data including fitness data of an individual, such as performance metrics and an instantaneous effort value, back to and/or from the server (computing module) 60. In some embodiments, the client 50 may include client applications or components executing on the client 50 for calibrating of a level of intensity of a fitness exercise, for an individual performing the fitness exercise, and the client 50 may communicate corresponding data to the server (e.g., computing module) 60.

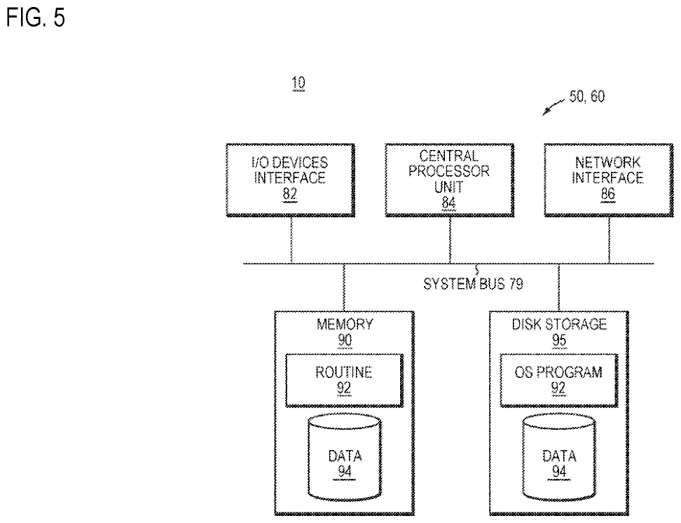

Some embodiments of the system 10 may include a computer system for calibrating of a level of intensity of a fitness exercise, for an individual performing the fitness exercise. The system 10 may include a plurality of processors 84. The system 10 may also include a memory 90. The memory 90 may include: (i) computer code instructions stored thereon; and/or (ii) data including fitness data of an individual, such as performance metrics and an instantaneous effort value. The data may include segments including portions of the performance metrics or instantaneous effort value. The memory 90 may be operatively coupled to the plurality of processors 84 such that, when executed by the plurality of processors 84, the computer code instructions may cause the computer system 10 to implement a computing module (the computing module being located on, in, or implemented by any of elements 50, 60, 70 of FIG. 4 or elements 82, 84, 86, 90, 92, 94, 95 of FIG. 5) configured to perform one or more functions.

According to some embodiments, FIG. 5 is a diagram of an example internal structure of a computer (e.g., client processor/device 50 or server computers 60) in the computer system 10 of FIG. 4. Each computer 50, 60 contains a system bus 79, where a bus is a set of hardware lines used for data transfer among the components of a computer or processing system. The system bus 79 is essentially a shared conduit that connects different elements of a computer system (e.g., processor, disk storage, memory, input/output ports, network ports, etc.) that enables the transfer of information between the elements. Attached to the system bus 79 is an I/O device interface 82 for connecting various input and output devices (e.g., keyboard, mouse, displays, printers, speakers, etc.) to the computer 50, 60. A network interface 86 allows the computer to connect to various other devices attached to a network (e.g., network 70 of FIG. 4). Memory 90 provides volatile storage for computer software instructions 92 and data 94 used to implement some embodiments (e.g., fitness data described herein). Disk storage 95 provides non-volatile storage for computer software instructions 92 and data 94 used to implement an embodiment of the present disclosure. A central processor unit 84 is also attached to the system bus 79 and provides for the execution of computer instructions.

In one embodiment, the processor routines 92 and data 94 are a computer program product (generally referenced 92), including a computer readable medium (e.g., a removable storage medium such as one or more DVD-ROM's, CD-ROM's, diskettes, tapes, etc.) that provides at least a portion of the software instructions for the present disclosure. The computer program product 92 can be installed by any suitable software installation procedure, as is well known in the art. In another embodiment, at least a portion of the software instructions may also be downloaded over a cable, communication and/or wireless connection. Other embodiments may include a computer program propagated signal product 107 (of FIG. 4) embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the routines/program 92 of the present disclosure.

In alternate embodiments, the propagated signal is an analog carrier wave or digital signal carried on the propagated medium. For example, the propagated signal may be a digitized signal propagated over a global network (e.g., the Internet), a telecommunications network, or other network. In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, storage medium and the like.

Embodiments or aspects thereof may be implemented in the form of hardware (including but not limited to hardware circuitry), firmware, or software. If implemented in software, the software may be stored on any non-transient computer readable medium that is configured to enable a processor to load the software or subsets of instructions thereof. The processor then executes the instructions and is configured to operate or cause an apparatus to operate in a manner as described herein.

Further, hardware, firmware, software, routines, or instructions may be described herein as performing certain actions and/or functions of the data processors. However, it should be appreciated that such descriptions contained herein are merely for convenience and that such actions in fact result from computing devices, processors, controllers, or other devices executing the firmware, software, routines, instructions, etc.

It should be understood that the flow diagrams, block diagrams, and network diagrams may include more or fewer elements, be arranged differently, or be represented differently. But it further should be understood that certain implementations may dictate the block and network diagrams and the number of block and network diagrams illustrating the execution of the embodiments be implemented in a particular way.

Accordingly, further embodiments may also be implemented in a variety of computer architectures, physical, virtual, cloud computers, and/or some combination thereof, and, thus, the data processors described herein are intended for purposes of illustration only and not as a limitation of the embodiments.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A processor-implemented method of calibrating, for an individual performing a fitness exercise, a level of intensity of the fitness exercise given a target effort value, the method comprising:
determining an instantaneous effort value corresponding to an instantaneous level of physical and mental exertion for the individual, the determining including:
obtaining measured data from an exertion monitor associated with the individual, the measured data including at least measurements of power;
determining performance metrics from the measured data, the performance metrics including at least a peak power value and an average power value;
obtaining historical performance metrics for the individual, the historical performance metrics including at least historical peak power values and historical average power values; and
calculating, using a polynomial equation derived from a best-fit analysis applied to a set of historical observed estimated effort values, the instantaneous effort value for the individual based on the determined and historical performance metrics;
communicating, via a display device, the instantaneous effort value to the individual;
determining a difference between the instantaneous effort value and the target effort value; and
adjusting an aspect of the fitness exercise based on the determined difference.

2. The method of claim 1 wherein the exertion monitor includes sensors placed proximal to the individual or to an exercise machine employed by the individual.

3. The method of claim 1 wherein the fitness exercise is one of running, cycling, and rowing.

4. The method of claim 1 wherein the performance metrics include measurements of at least one of cadence, a distance-based parameter, heart rate, respiratory rate, and body positioning.

5. The method of claim 1 wherein the display device is disposed proximal to an exercise machine.

6. The method of claim 1 wherein communicating the instantaneous effort value to the individual includes indicating, on the display device, a visual representation of the instantaneous effort value, or a time series thereof, during performance of the fitness exercise by the individual.

7. The method of claim 1 wherein adjusting an aspect of the fitness exercise includes automatically modifying a level of resistance on an exercise machine.

8. The method of claim 1 wherein historical performance metrics, including average power values, are modified by a decay factor such that more recent historical performance metrics are weighted more strongly and less recent historical performance metrics are weighted less strongly in a calculation of the instantaneous effort value.

9. The method of claim 1 further comprising transmitting an audio and video representation of the individual's performance of the fitness exercise to a device of a remotely located trainer, and further comprising transmitting an audio and video representation of a coaching session of the trainer to the display device to be viewed by the individual.

10. A system for calibrating, for an individual performing a fitness exercise, a level of intensity of the fitness exercise given a target effort value, the system comprising a processor and a memory device with instructions loaded thereon, the instructions, when loaded, configuring the processor to:
determine an instantaneous effort value corresponding to an instantaneous level of physical and mental exertion for the individual, the determining including:
obtaining measured data from an exertion monitor associated with the individual, the measured data including at least measurements of power;
determining performance metrics from the measured data, the performance metrics including at least a peak power value and an average power value;
obtaining historical performance metrics for the individual, the historical performance metrics including at least historical peak power values and historical average power values; and
calculating, using a polynomial equation derived from a best-fit analysis applied to a set of historical observed estimated effort values, the instantaneous effort value for the individual based on the determined and historical performance metrics;
communicate, via a display device, the instantaneous effort value to the individual;
determine a difference between the instantaneous effort value and the target effort value; and
adjust an aspect of the fitness exercise based on the determined difference.

11. The system of claim 10 wherein the exertion monitor includes sensors placed proximal to the individual or to an exercise machine employed by the individual.

12. The system of claim 10 wherein the fitness exercise is one of running, cycling, and rowing.

13. The system of claim 10 wherein the performance metrics include measurements of at least one of cadence, a distance-based parameter, heart rate, respiratory rate, and body positioning.

14. The system of claim 10 wherein the display device is disposed proximal to an exercise machine.

15. The system of claim 10 wherein the communicating of the instantaneous effort value to the individual includes indicating, on the display device, a visual representation of the instantaneous effort value, or a time series thereof, during performance of the fitness exercise by the individual.

16. The system of claim 10 wherein the adjusting of an aspect of the fitness exercise includes automatically modifying a level of resistance on an exercise machine.

17. The system of claim 10 wherein historical performance metrics including average power values are modified by a decay factor such that more recent historical performance metrics are weighted more strongly and less recent historical performance metrics are weighted less strongly in a calculation of the instantaneous effort value.

18. The system of claim 10 wherein the processor is further configured to transmit an audio and video representation of the individual's performance of the fitness exercise to a device of a remotely located trainer, and to transmit an audio and video representation of a coaching session of the trainer to the display device to be viewed by the individual.
